(19) United States
(12) Patent Application Publication
(10) Pub. No.: US 2024/0294750 A1
(43) Pub. Date: Sep. 5, 2024

(54) POLYCARBONATE COMPOSITIONS

(71) Applicant: BASF SE, Ludwigshafen Am Rhein (DE)

(72) Inventors: Stephen Mark Andrews, New Fairfield, CT (US); Markus Grob, Windisch (CH)

(73) Assignee: BASF SE, Ludwigshafen Am Rhein (DE)

(21) Appl. No.: 18/647,000

(22) Filed: Apr. 26, 2024

Related U.S. Application Data
(62) Division of application No. 16/763,160, filed on May 11, 2020, now abandoned.
(60) Provisional application No. 62/585,826, filed on Nov. 14, 2017.

(51) Int. Cl. C08L 69/00; C08K 5/00; C08K 5/1535; C08K 5/5377; C08K 5/5393; C08K 5/5397

(52) U.S. Cl. CPC C08L 69/00; C08K 5/005; C08K 5/1535; C08K 5/5377; C08K 5/5393; C08K 5/5397; C08L 2666/34

(57) ABSTRACT

Polycarbonate compositions comprising one or more additives selected from the group consisting of benzofuranone stabilizers of formula I, wherein R₁, R₂, R₃, R₄ and R₅ are each independently hydrogen, halogen, hydroxy, C₁-C₂₅alkyl, C₂-C₂₅alkenyl, C₂-C₂₅alkynyl, C₇-C₉phenylalkyl, phenyl, C₅-C₈cycloalkyl, C₁-C₅galkoxy, C₁-C₈alkylamine, di(C₁-C₈alkyl)amino, C₁-C₂₅alkanoyloxy, C₁-C₂₅alkanoylamino, C₃-C₂₅alkenoyloxy, C₃-C₂₅alkanoyloxy interrupted by one to three —O—, —S—, —NH— or —N(C₁-C₈alkyl)-, wherein C₁-C₂₅alkyl, C₂-C₂₅alkenyl, C₂-C₂₅alkynyl, C₇-C₉phenylalkyl, phenyl, C₅-C₅cycloalkyl, C₁-C₁₅alkoxy, C₁-C₅alkylamine, di(C₁-C₈alkyl)amino, C₁-C₂₅alkanoyloxy, C₁-C₂₅alkanoylamino, C₃-C₂₅alkenoyloxy and interrupted C₃-C₂₅alkenoyloxy are unsubstituted or substituted by one to three groups selected from halogen, hydroxy, thiol, amino, C₁-C₄alkylamino, di(C₁-C₄alkyl)amino and C₁-C₄alkyl.

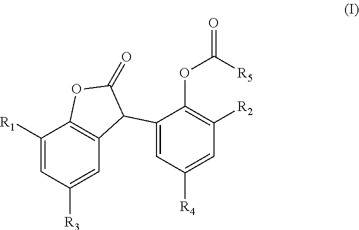

### BACKGROUND

Polycarbonates are desired thermoplastic polymers for their strong mechanical properties and excellent optical properties including high light transmittance, high gloss and clarity. Polycarbonates will, however, undergo yellowing and loss of optical and mechanical properties when subjected to cyclical or constant elevated temperatures.

Polycarbonates are employed in applications such as solar energy control window films for construction, automotive and commercial lamp covers, reflective housings for LED lighting, and light diffuser films for LED and LCD electronic display monitors. These applications are subject to elevated temperatures.

There exists a need to more thoroughly protect polycarbonates from the deleterious effects of heat, light and oxygen, which effects result in yellowing, loss of transparency and loss of gloss. Generally, conventional hindered phenolic antioxidants and/or organic phosphite or phosphine stabilizers are employed in polycarbonate.

### SUMMARY

Disclosed is a composition comprising a polycarbonate and one or more additives selected from the group consisting of benzofuranone stabilizers of formula I

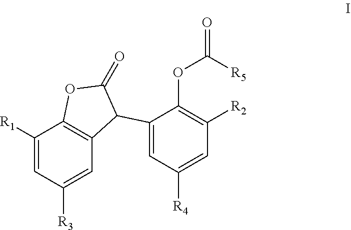

wherein R₁, R₂, R₃, R₄ and R₅ are each independently hydrogen, halogen, hydroxy, C₁-C₂₅alkyl, C₂-C₂₅alkenyl, C₂-C₂₅alkynyl, C₇-C₉phenylalkyl, phenyl, C₅-C₈cycloalkyl, C₁-C₁₈alkoxy, C₁-C₅alkylamine, di(C₁-C₅alkyl)amino, C₁-C₂₅alkanoyloxy, C₁-C₂₅alkanoylamino, C₃-C₂₅alkenoyloxy or C₃-C₂₅alkanoyloxy interrupted by one to three —O—, —S—, —NH— or —N(C-C₈alkyl)-, wherein C₁-C₂₅alkyl, C₂-C₂₅alkenyl, C₂-C₂₅alkynyl, C₇-C₉phenylalkyl, phenyl, C₅-C₈cycloalkyl, C₁-C₁₈alkoxy, C₁-C₅alkylamine, di(C₁-C₅alkyl)amino, C₁-C₂₅alkanoyloxy, C₁-C₂₅alkanoylamino, C₃-C₂₅alkenoyloxy and interrupted C₃-C₂₅alkenoyloxy are unsubstituted or substituted by one to three groups selected from halogen, hydroxy, thiol, amino, C₁-C₄alkylamino, di(C₁-C₄alkyl)amino and C₁-C₄alkyl.

Also disclosed is a method of stabilizing polycarbonate against the deleterious effects of heat, light and oxygen, the method comprising incorporating into a polycarbonate one or more additives selected from the group consisting of benzofuranone stabilizers of formula I.

Also disclosed is an additive composition comprising one or more benzofuranone stabilizers of formula I and one or more organophosphorus stabilizers.

### DETAILED DISCLOSURE

Polycarbonates may be obtained from bisphenol A and phosgene or a phosgene analogue such as trichloromethylchloroformate, triphosgene or diphenylcarbonate by condensation, in the latter case usually with addition of a suitable transesterification catalyst, for example a boron hydride, an amine, e.g. 2-methylimidazole, or a quaternary ammonium salt. In addition to bisphenol A, other bisphenol components may also be used, and it is also possible to use monomers which are halogenated in the benzene nucleus. Suitable bisphenol components include for example 2,2-bis(4′-hydroxyphenyl)propane (bisphenol A), 2,4′-dihydroxydiphenylmethane, bis(2-hydroxyphenyl)methane, bis(4-hydroxyphenyl)methane, bis-(4-hydroxy-5-propylphenyl)methane, 1,1-bis(4′-hydroxyphenyl)ethane, bis(4-hydroxyphenyl)cyclohexylmethane, 2,2-bis(4′-hydroxyphenyl)-1-phenylpropane, 2,2-bis(3′, 5′-dimethyl-4′-hydroxyphenyl)propane, 2,2-bis(3′, 5′-dibromo-4′-hydroxyphenyl)propane, 2,2-bis(3′,5′-dichloro-4′-hydroxyphenyl)propane, 1,1-bis(4′-hydroxyphenyl)cyclododecane, 1,1-bis(3′5, ′-dimethyl-4′-hydroxyphenyl)cyclododecane, 1,1-bis(4′-hydroxyphenyl)-3,3,5-trimethylcyclohexane, 1,1-bis(4′-hydroxyphenyl)-3,3,5,5-tetramethylcyclohexane and 1,1-bis(4′-hydroxyphenyl)-3,3,5-trimethylcyclopentane. In addition to bisphenols, polycarbonates may be obtained from other diols, for example dianhydrohexitols including isosorbide, isoidide and isomannide. Polycarbonates may employ one or more of the aforementioned diols as well as further diols. Polycarbonates may be branched by suitable amounts of more than difunctional monomers.

In some embodiments, polycarbonate may be present in compositions of the disclosure in a major amount, for example from any one of about 55 wt %, about 60 wt %, about 65 wt %, about 70 wt %, about 75 wt % or about 80 wt % to any one of about 85 wt %, about 90 wt %, about 95 wt %, about 96 wt %, about 97 wt %, about 98 wt % or about 99 wt %.

Polycarbonate may be in the form of sheets, films or molded parts. Polycarbonate compositions may be employed for instance in exterior materials of electrical and electronic products, automobile components, building materials, optical components, etc.

In some embodiments, polycarbonate compositions may be employed in automotive headlamps, street lamps, solar energy control window films for construction (buildings), reflective housings for LED lighting, light diffusion films for LED and LCD electronic display monitors, back-lighting films in computer and large console display screens and containers such as bottles. Automotive includes passenger cars, trucks, vans, construction vehicles, sport utility vehicles, motorcycles and the like.

Polycarbonate articles may also include windowpanes, greenhouse panes, roofing articles, mirrors, goggles, noise-absorbing walls, windshields (e.g. motorcycle windshields), solar cell cover or substrate, display covers, touch panels or screens, parts for recreational machines (e.g. pinball machines) and the like.

Polycarbonate (PC) films may for instance have a thickness of from less than about 0.05 mm to about 0.50 mm, for instance from any of about 0.05 mm, 0.07 mm, 0.09 mm, 0.10 mm, 0.15 mm, about 0.20 mm or about 0.25 mm to any of about 0.30 mm, about 0.35 mm, about 0.40 mm, about 0.45 mm or about 0.50 mm.

Polycarbonate sheet may for instance have a thickness of from about 0.5 mm to greater than about 3.1 mm. PC sheet may be thicker, for example for ballistic window sheet. PC sheet may be from any of about 0.5 mm, about 0.7 mm, about 1.0 mm, about 1.5 mm or about 2.0 mm to any of about 2.5 mm, about 2.7 mm or about 3.1 mm or greater.

Both film and sheet may be mono- or multi-layer and may include layers comprising other polymers or plastics. Films and sheets may be prepared via extrusion or co-extrusion methods.

In certain embodiments, the polycarbonate compositions may be transparent to light having a wavelength of from any of about 350 nm, about 360 nm, about 370 nm, about 380 nm, about 390 nm, about 400 nm, about 410 nm, about 420 nm, about 430 nm, about 440 nm or about 450 nm to about 700 nm. In some embodiments, the polycarbonate compositions are transparent to visible light (about 390 nm to about 700 nm). In some embodiments, transparent means that ≥90%, ≥91%, ≥92%, ≥93%, ≥94%, ≥95%, ≥96%, ≥97%, ≥98% or ≥99% of light may pass through the composition.

The benzofuranones of the invention include compounds of formula I

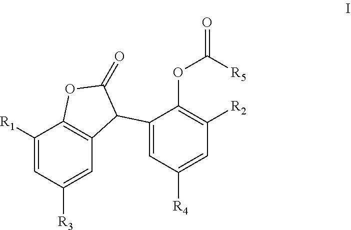

wherein R₁, R₂, R₃, R₄ and R₅ are each independently hydrogen, halogen, hydroxy, C₁-C₂₅alkyl, C₂-C₂₅alkenyl, C₂-C₂₅alkynyl, C₇-C₉phenylalkyl, phenyl, C₅-C₅cycloalkyl, C₁-C₁₈alkoxy, C₁-C₅alkylamine, di(C₁-C₅alkyl)amino, C₁-C₂₅alkanoyloxy, C₁-C₂₅alkanoylamino, C₃-C₂₅alkenoyloxy or C₃-C₂₅alkanoyloxy interrupted by one to three —O—, —S—, —NH— or —N(C-C₈alkyl)-, wherein Cr₁-C₂₅alkyl, C₂-C₂₅alkenyl, C₂-C₂₅alkynyl, C₇-C₉phenylalkyl, phenyl, C₅-C₈cycloalkyl, C₁-C₁₈alkoxy, C₁-C₈alkylamine, di(C₁-C₈alkyl)amino, Cr₁-C₂₅alkanoyloxy, C-C₂₅alkanoylamino, C₃-C₂₅alkenoyloxy and interrupted C₃-C₂₅alkenoyloxy are unsubstituted or substituted by one to three groups selected from halogen, hydroxy, thiol, amino, C₁-C₄alkylamino, di(C₁-C₄alkyl)amino and C₁-C₄alkyl.

In certain embodiments, R₁ and R₂ are independently hydrogen or C₁-C₈alkyl, R₃ and R₄ are independently C₁-C₁₂alkyl and R₅ is C₁-C₈alkyl, phenyl, C₁-C₅alkylphenyl, 3,5-di(C-C₈alkyl)-4-hydroxyphenyl or C₁-C₈alkyl(3,5-di(C₁-C₈alkyl)-4-hydroxyphenyl).

In certain embodiments, R₁ and R₂ are hydrogen and R₃ and R₄ are C₁-C₈alkyl. In some embodiments, R₅ is C₁-C₄alkyl, phenyl, C₁-C₄alkylphenyl, 3,5-di(C₁-C₄alkyl)-4-hydroxyphenyl or C₁-C₄alkyl(3,5-di(C₁-C₄alkyl)-4-hydroxyphenyl.

Alkyl groups may be linear or branched and may include methyl, ethyl, propyl, isopropyl, n-butyl, sec-butyl, isobutyl, tert-butyl, 2-ethylbutyl, n-pentyl, isopentyl, 1-methylpentyl, 1,3-dimethylbutyl, n-hexyl, 1-methylhexyl, n-heptyl, isoheptyl, 1,1,3,3-tetramethylbutyl, 1-methylheptyl, 3-methylheptyl, n-octyl, tert-octyl, 2-ethylhexyl, 1,1,3-trimethylhexyl, 1,1,3,3-tetramethylpentyl, nonyl, decyl, undecyl, 1-methylundecyl and dodecyl.

Cycloalkyl includes cyclohexyl, cyclopentyl and cycloheptyl. Halogen is fluoro, chloro, bromo or iodo. Phenylalkyl includes benzyl and cumyl. Alkenyl and alkynyl are unsaturated versions of alkyl. Alkyl portions of for example alkoxy may be defined as for alkyl.

In certain embodiments, benzofuranones of the disclosure include compounds of formula

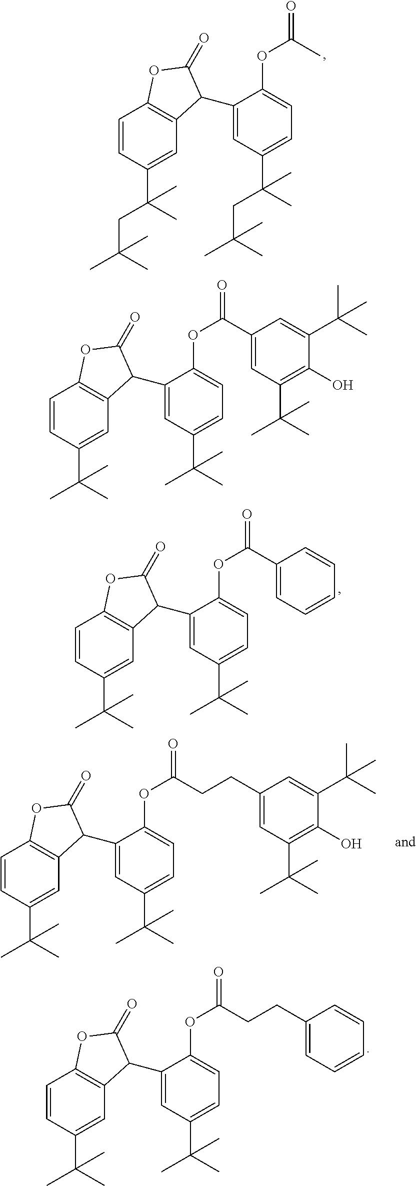

Benzofuranones of formula I may be prepared as described in U.S. Pat. Nos. 6,881,774 and 8,840,810.

Polycarbonate compositions may include one or more ultraviolet light absorbers (UVAs). In some embodiments, UV light absorbers may be selected from the group consisting of hydroxyphenylbenzotriazole, tris-aryl-s-triazine, hydroxybenzoate, 2-hydroxybenzophenone and cyanoacrylate ultraviolet light absorbers.

The hydroxyphenylbenzotriazole UVAs are for instance disclosed in U.S. Pat. Nos. 3,004,896; 3,055,896; 3,072,585; 3,074,910; 3,189,615; 3,218,332; 3,230,194; 4,127,586; 4,226,763; 4,275,004; 4,278,589; 4,315,848; 4,347,180; 4,383,863; 4,675,352; 4,681,905, 4,853,471; 5,268,450; 5,278,314; 5,280,124; 5,319,091; 5,410,071; 5,436,349; 5,516,914; 5,554,760; 5,563,242; 5,574,166; 5,607,987, 5,977,219 and 6,166,218, such as 2-(2-hydroxy-5-methylphenyl)-2H-benzotriazole, 2-(3,5-di-t-butyl-2-hydroxyphenyl)-2H-benzotriazole, 2-(2-hydroxy-5-t-butylphenyl)-2H-benzotriazole, 2-(2-hydroxy-5-t-octylphenyl)-2H-benzotriazole, 5-chloro-2-(3,5-di-t-butyl-2-hydroxyphenyl)-2H-benzotriazole, 5-chloro-2-(3-t-butyl-2-hydroxy-5-methylphenyl)-2H-benzotriazole, 2-(3-sec-butyl-5-t-butyl-2-hydroxyphenyl)-2H-benzotriazole, 2-(2-hydroxy-4-octyloxyphenyl)-2H-benzotriazole, 2-(3,5-di-t-amyl-2-hydroxyphenyl)-2H-benzotriazole, 2-(3,5-bis-α-cumyl-2-hydroxyphenyl)-2H-benzotriazole, 2-(3-t-butyl-2-hydroxy-5-(2-(ω-hydroxy-octa-(ethyleneoxy)carbonyl-ethyl)-, phenyl)-2H-benzotriazole, 2-(3-dodecyl-2-hydroxy-5-methylphenyl)-2H-benzotriazole, 2-(3-t-butyl-2-hydroxy-5-(2-octyloxycarbonyl)ethylphenyl)-2H-benzotriazole, dodecylated 2-(2-hydroxy-5-methylphenyl)-2H-benzotriazole, 2-(3-t-butyl-2-hydroxy-5-(2-octyloxycarbonylethyl)phenyl)-5-chloro-2H-benzotriazole, 2-(3-tert-butyl-5-(2-(2-ethylhexyloxy)-carbonylethyl)-2-hydroxyphenyl)-5-chloro-2H-benzotriazole, 2-(3-t-butyl-2-hydroxy-5-(2-methoxycarbonylethyl)phenyl)-5-chloro-2H-benzotriazole, 2-(3-t-butyl-2-hydroxy-5-(2-methoxycarbonylethyl)phenyl)-2H-benzotriazole, 2-(3-t-butyl-5-(2-(2-ethylhexyloxy)carbonylethyl)-2-hydroxyphenyl)-2H-benzotriazole, 2-(3-t-butyl-2-hydroxy-5-(2-isooctyloxycarbonylethyl)phenyl-2H-benzotriazole, 2,2′-methylene-bis(4-t-octyl-(6-2H-benzotriazol-2-yl)phenol), 2-(2-hydroxy-3-α-cumyl-5-t-octylphenyl)-2H-benzotriazole, 2-(2-hydroxy-3-t-octyl-5-α-cumylphenyl)-2H-benzotriazole, 5-fluoro-2-(2-hydroxy-3,5-di-α-cumyl-phenyl)-2H-benzotriazole, 5-chloro-2-(2-hydroxy-3,5-di-α-cumylphenyl)-2H-benzotriazole, 5-chloro-2-(2-hydroxy-3-α-cumyl-5-t-octylphenyl)-2H-benzotriazole, 2-(3-t-butyl-2-hydroxy-5-(2-isooctyloxycarbonylethyl)phenyl)-5-chloro-2H-benzotriazole, 5-trifluoromethyl-2-(2-hydroxy-3-α-cumyl-5-t-octylphenyl)-2H-benzotriazole, 5-trifluoromethyl-2-(2-hydroxy-5-t-octylphenyl)-2H-benzotriazole, 5-trifluoromethyl-2-(2-hydroxy-3,5-di-t-octylphenyl)-2H-benzotriazole, methyl 3-(5-trifluoromethyl-2H-benzotriazol-2-yl)-5-t-butyl-4-hydroxy-hydrocinnamate, 5-butylsulfonyl-2-(2-hydroxy-3-α-cumyl-5-t-octylphenyl)-2H-benzotriazole, 5-trifluoromethyl-2-(2-hydroxy-3-ca-cumyl-5-t-butylphenyl)-2H-benzotriazole, 5-trifluoromethyl-2-(2-hydroxy-3,5-di-t-butylphenyl)-2H-benzotriazole, 5-trifluoromethyl-2-(2-hydroxy-3,5-di-α-cumylphenyl)-2H-benzotriazole, 5-butylsulfonyl-2-(2-hydroxy-3,5-di-t-butyl-phenyl)-2H-benzotriazole and 5-phenylsulfonyl-2-(2-hydroxy-3,5-di-t-butylphenyl)-2H-benzotriazole.

Tris-aryl-s-triazine UVAs are for instance those disclosed in U.S. Pat. Nos. 3,843,371; 4,619,956; 4,740,542; 5,096,489; 5,106,891; 5,298,067; 5,300,414; 5,354,794; 5,461,151; 5,476,937; 5,489,503; 5,543,518; 5,556,973; 5,597,854; 5,681,955; 5,726,309; 5,736,597; 5,942,626; 5,959,008; 5,998,116; 6,013,704; 6,060,543; 6,242,598 and 6,255,483, for example 4,6-bis-(2,4-dimethylphenyl)-2-(2-hydroxy-4-octyloxyphenyl)-s-triazine, CYASORB 1164, 4,6-bis-(2,4-dimethylphenyl)-2-(2,4-dihydroxyphenyl)-s-triazine, 2,4-bis(2,4-dihydroxyphenyl)-6-(4-chlorophenyl)-s-triazine, 2,4-bis[2-hydroxy-4-(2-hydroxyethoxy)phenyl]-6-(4-chlorophenyl)-s-triazine, 2,4-bis[2-hydroxy-4-(2-hydroxy-4-(2-hydroxyethoxy)phenyl]-6-(2,4-dimethylphenyl)-s-triazine, 2,4-bis[2-hydroxy-4-(2-hydroxyethoxy)phenyl]-6-(4-bromophenyl)-s-triazine, 2,4-bis[2-hydroxy-4-(2-acetoxyethoxy)phenyl]-6-(4-chlorophenyl)-s-triazine, 2,4-bis(2,4-dihydroxyphenyl)-6-(2,4-dimethylphenyl)-s-triazine, 2,4-bis(4-biphenylyl)-6-(2-hydroxy-4-octyloxycarbonylethylidene-oxyphenyl)-s-triazine, 2,4-bis(4-biphenylyl)-6-(2-hydroxy-4-(2-ethylhexyl)oxyphenyl)-s-triazine, 2-phenyl-4-[2-hydroxy-4-(3-sec-butyloxy-2-hydroxypropyloxy)phenyl]-6-[2-hydroxy-4-(3-sec-amyloxy-2-hydroxypropyloxy)phenyl]-s-triazine, 2,4-bis(2,4-dimethylphenyl)-6-[2-hydroxy-4-(3-benzyloxy-2-hydroxypropyloxy)phenyl]-s-triazine, 2,4-bis(2-hydroxy-4-n-butyloxyphenyl)-6-(2,4-di-n-butyloxyphenyl)-s-triazine, 2,4-bis(2,4-dimethylphenyl)-6-[2-hydroxy-4-(3-nonyloxy*-2-hydroxypropyloxy)-5-α-cumylphenyl]-s-triazine (* denotes a mixture of octyloxy, nonyloxy and decyloxy groups), methylenebis-{2,4-bis(2,4-dimethyl-phenyl)-6-[2-hydroxy-4-(3-butyloxy-2-hydroxypropoxy)phenyl]-s-triazine}, methylene bridged dimer mixture bridged in the 3:5′, 5:5′ and 3:3′ positions in a 5:4:1 ratio, 2,4,6-tris(2-hydroxy-4-isooctyloxycarbonylisopropylideneoxyphenyl)-s-triazine, 2,4-bis(2,4-dimethylphenyl)-6-(2-hydroxy-4-hexyloxy-5-α-cumylphenyl)-s-triazine, 2-(2,4,6-trimethylphenyl)-4,6-bis[2-hydroxy-4-(3-butyloxy-2-hydroxypropyloxy)phenyl]-s-triazine, 2,4,6-tris[2-hydroxy-4-(3-sec-butyloxy-2-hydroxypropyloxy)phenyl]-s-triazine, mixture of 4,6-bis-(2,4-dimethylphenyl)-2-(2-hydroxy-4-(3-dodecyloxy-2-hydroxypropoxy)-phenyl)-s-triazine and 4,6-bis-(2,4-dimethylphenyl)-2-(2-hydroxy-4-(3-tridecyloxy-2-hydroxypropoxy)-phenyl)-s-triazine, TINUVIN 400, 4,6-bis-(2,4-dimethylphenyl)-2-(2-hydroxy-4-(3-(2-ethylhexyloxy)-2-hydroxypropoxy)-phenyl)-s-triazine and 4,6-diphenyl-2-(4-hexyloxy-2-hydroxyphenyl)-s-triazine.

Hydroxybenzoate UV absorbers are for instance esters of substituted and unsubstituted benzoic acids, as for example 4-tert-butylphenyl salicylate, phenyl salicylate, octylphenyl salicylate, dibenzoyl resorcinol, bis(4-tert-butylbenzoyl) resorcinol, benzoyl resorcinol, 2,4-di-tert-butylphenyl 3,5-di-tert-butyl-4-hydroxybenzoate, hexadecyl 3,5-di-tert-butyl-4-hydroxybenzoate, octadecyl 3,5-di-tert-butyl-4-hydroxybenzoate, 2-methyl-4,6-di-tert-butylphenyl 3,5-di-tert-butyl-4-hydroxybenzoate.

2-Hydroxybenzophenone UV absorbers are for example the 4-hydroxy, 4-methoxy, 4-octyloxy, 4-decyloxy, 4-dodecyloxy, 4-benzyloxy, 4,2′,4′-trihydroxy and 2′-hydroxy-4,4′-dimethoxy derivatives.

Cyanoacrylate UV absorbers are for instance pentaerythritol tetrakis(2-cyano-3,3-diphenylacrylate), α-cyano-β,β-diphenylacrylic acid ethyl ester or isooctyl ester and α-cyano-β-methyl-p-methoxy-cinnamic acid methyl ester or butyl ester.

Many of the UVAs are commercial, for example TINUVIN 326, TINUVIN 360, TINUVIN 234, TINUVIN 1577, TINUVIN 1600, CYASORB UV 1164, CYASORB THT, CYASORB UV 2908, CHIMASSORB 81, UVINUL 3030, etc.

In some embodiments, ultraviolet light absorbers may be present, in total, from any of about 0.01 weight percent (wt %), about 0.05 wt %, about 0.1 wt %, about 0.2 wt %, about 0.5 wt %, about 0.7 wt %, about 1.0 wt % or about 1.2 wt % to any of about 1.4 wt %, about 1.6 wt %, about 1.8 wt %, about 2.0 wt %, about 2.3 wt %, about 2.5 wt %, about 2.7 wt % or about 3.0 wt %, based on the weight of the polycarbonate.

In certain embodiments, in a multi-layer film or sheet, an interior bulk layer and a top or cap layer may comprise different UVAs.

The polycarbonate compositions may optionally contain one or more organophosphorus stabilizers. Organic phosphorus stabilizers include organic phosphite and organic phosphonite stabilizers and include for example triphenyl phosphite, diphenyl alkyl phosphites, phenyl dialkyl phosphites, tris(nonylphenyl) phosphite, trilauryl phosphite, trioctadecyl phosphite, distearyl pentaerythritol diphosphite (F), tris(2,4-di-tert-butylphenyl) phosphite, bis(2,4-di-α-cumylphenyl) pentaerythrtitol diphosphite (K), diisodecyl pentaerythritol diphosphite, bis(2,4-di-tert-butylphenyl) pentaerythritol diphosphite (D), bis(2,6-di-tert-butyl-4-methylphenyl) pentaerythritol diphosphite (E), bisisodecyloxy-pentaerythritol diphosphite, bis(2,4-di-tert-butyl-6-methylphenyl) pentaerythritol diphosphite, bis(2,4,6-tri-tert-butylphenyl) pentaerythritol diphosphite, tristearyl sorbitol triphosphite, tetrakis (2,4-di-tert-butylphenyl) 4,4′-biphenylene-diphosphonite (H), 6-isooctyloxy-2,4,8,10-tetra-tert-butyl-dibenzo[d,f][1,3,2]dioxaphosphepin (C), 6-fluoro-2,4,8,10-tetra-tert-butyl-12-methyl-dibenzo[d,g][1,3,2]dioxaphosphocin (A), bis(2,4-di-tert-butyl-6-methylphenyl) methyl phosphite, bis(2,4-di-tert-butyl-6-methylphenyl) ethyl phosphite (G), 2,2′,2″-nitrilo[triethyltris(3,3′5,5′-tetra-tert-butyl-1,1′-biphenyl-2,2′-diyl) phosphite] (B), bis(2,4-di-t-butylphenyl) octylphosphite, poly(4,4′-{2,2′-dimethyl-5,5′-di-t-butylphenylsulfide-}octylphosphite), poly(4,4′{-isopropylidenediphenol}-octylphosphite), poly(4,4′-{isopropylidenebis[2,6-dibromophenol]}-octylphosphite), poly(4,4′-{2,2′-dimethyl-5,5′-di-t-butylphenylsulfide}-pentaerythrityl diphosphite),

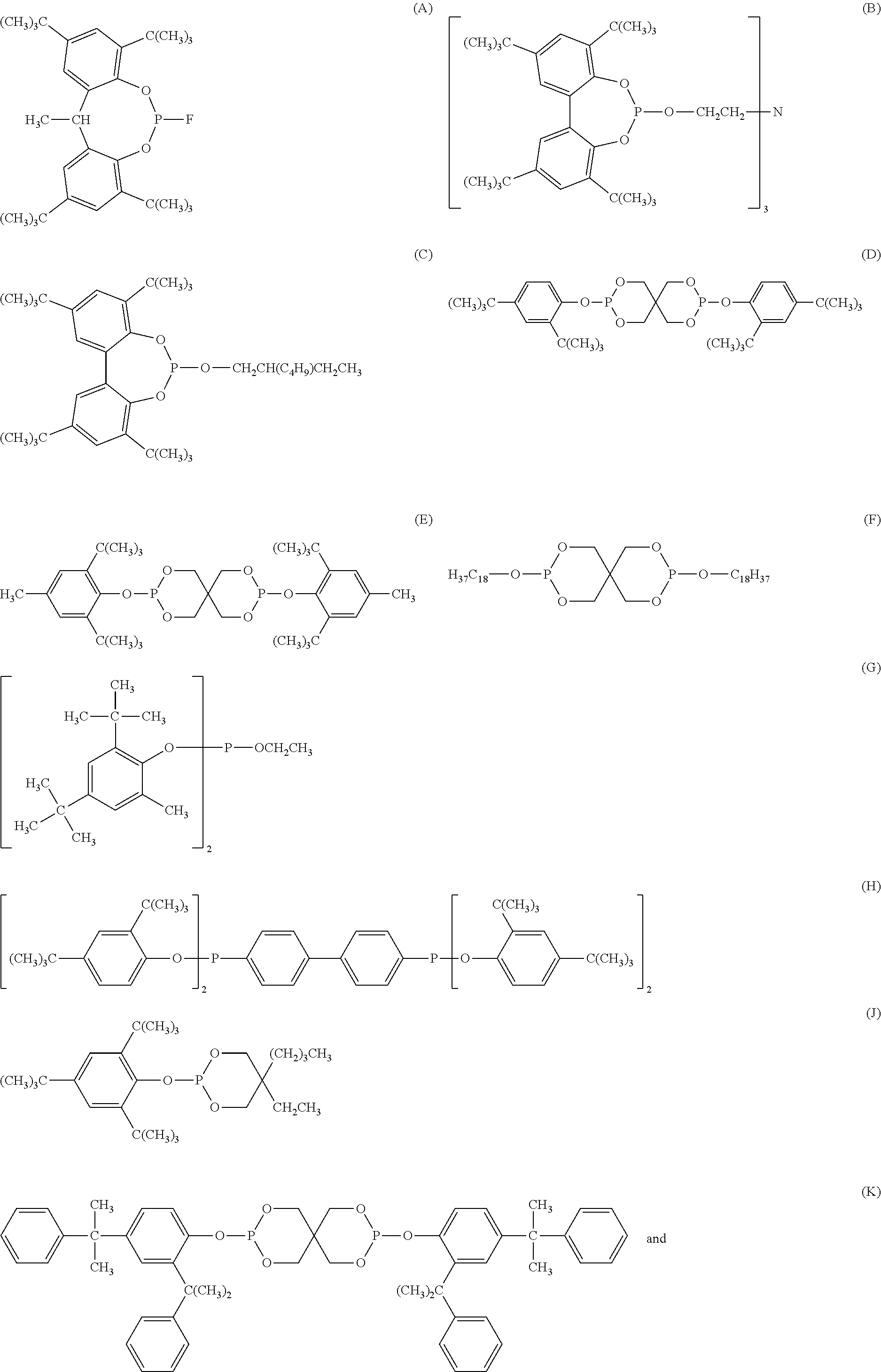

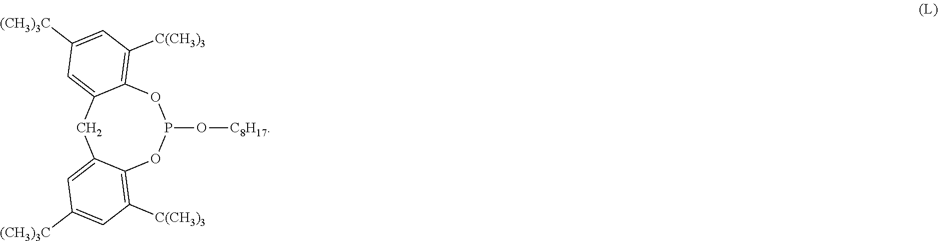

In some embodiments, organophosphorus stabilizers also include organic phosphine stabilizers. Organic phosphine stabilizers may be of formula P(R₁)(R₂)(R₃), wherein R₁, R₂ and R₃ are each independently H, C₁-C₂₀alkyl, C₃-C₇cycloalkyl, C₂-C₆alkenyl, phenyl or naphthyl, each unsubstituted or substituted by 1 to 3 hydroxy, amino, acetoxy, C₁-C₄alkoxy, C₁-C₁₀alkyl, C₁-C₄alkylcarbonyl, phenyl or C₁-C₄alkoxyphenyl.

In some embodiments, organic phosphine stabilizers may be selected from triethylphosphine, triisopropylphosphine, tri-n-butylphosphine, tricyclohexylphosphine, allyldiphenylphosphine, triphenylphosphine, diphenylphosphine, tri-2,4-dimethylphenylphosphine, tri-2,4,6-trimethylphenylphosphine, tri-o-tolylphosphine, tri-o-anisylphosphine, diphenylbutylphosphine, diphenyl-octadecylphosphine, tris-(p-nonylphenyl)-phosphine, tris-naphthylphosphine, diphenyl-(hydroxymethyl)-phosphine, diphenyl-acetoxymethylphosphine, diphenyl-(beta-ethylcarboxyethyl)phosphine, diphenyl-benzylphosphine, diphenyl-(p-hydroxyphenyl)-phosphine, diphenyl-1,4-dihydroxyphenyl-2-phosphine and phenyl-naphthyl-benzylphosphine.

The polycarbonate compositions may optionally contain one or more hindered phenolic antioxidants. Hindered phenolic antioxidants include for example tris(3,5-di-tert-butyl-4-hydroxybenzyl) isocyanurate, 1,3,5-tris-(3,5-di-tert-butyl-4-hydroxybenzyl)-2,4,6-trimethylbenzene, the calcium salt of the monoethyl ester of 3,5-di-tert-butyl-4-hydroxybenzylphosphonic acid, pentaerythritol tetrakis[3-(3,5-di-tert-butyl-4-hydroxyphenyl) propionate] and octadecyl 3-(3,5-di-tert-butyl-4-hydroxyphenyl) propionate.

In some embodiments, both an organic phosphorus stabilizer and a hindered phenolic antioxidant may be present, for example tris(2,4-di-tert-butylphenyl) phosphite and pentaerythritol tetrakis[3-(3,5-di-tert-butyl-4-hydroxyphenyl) propionate] and/or octadecyl 3-(3,5-di-tert-butyl-4-hydroxyphenyl) propionate.

In some embodiments, the benzofuranones of the disclosure are incorporated into a polycarbonate at levels of from any one of about 10 ppm, about 25 ppm, about 50 ppm, about 100 ppm about 150 ppm, about 175 ppm about 200 ppm, about 225 ppm, about 250 ppm, about 275 ppm, about 300 ppm, about 325 ppm, about 350 ppm, about 400 ppm, about 450 ppm or about 500 ppm to any one of about 550 ppm, about 600 ppm, about 650 ppm, about 700 ppm, about 750 ppm, about 800 ppm, about 850 ppm, about 900 ppm, about 950 ppm or about 1000 ppm (0.1 weight percent (wt %)) by weight, based on the weight of the polycarbonate.

In other embodiments, benzofuranones of the disclosure are incorporated into a polycarbonate at levels of from any one of about 0.1 wt %, about 0.5 wt %, about 0.75 wt %, about 1.0 wt %, about 1.5 wt %, about 2.0 wt %, about 2.5 wt %, about 3.0 wt %, about 3.5 wt % or about 4.0 wt % to any one of about 4.5 wt %, about 5.0 wt %, about 5.5 wt %, about 6.0 wt %, about 6.5 wt % or about 7.0 wt % (weight percent), based on the weight of the polycarbonate.

Organic phosphorus stabilizers are, when present, employed at the weight levels as outlined for the benzofuranones. In some embodiments, one or more benzofuranones and one or more organic phosphorus compounds may be employed in a weight/weight ratio of from any one of about 1/99, about 1/90, about 1/80, about 1/70, about 1/60, about 1/50, about 1/40, about 1/30, about 1/20, about 1/10, about 1/9, about 1/8, about 1/7, about 1/6, about 1/5, about 1/4, about 1/3, about 1/2 or about 1/1 to any one of about 2/1, about 3/1, about 4/1, about 5/1, about 6/1, about 7/1, about 8/1, about 9/1, about 10/1, about 20/1, about 30/1, about 40/1, about 50/1, about 60/1, about 70/1, about 80/1, about 90/1 or about 99/1.

In some embodiments, organic phosphorus stabilizers are, when present, employed at equal or higher weight levels than the one or more benzofuranones. For example, in some embodiments, the weight/weight ratio of the one or more benzofuranones to one or more organophosphorus stabilizers may be from any one of about 0.05, about 0.10, about 0.11, about 0.12, about 0.13, about 0.14, about 0.15, about 0.16, about 0.20, about 0.25, about 0.30, about 0.35, about 0.40, about 0.45 or about 0.50 to any one of about 0.55, about 0.60, about 0.65, about 0.70, about 0.75, about 0.80, about 0.85, about 0.90, about 0.95 or about 1.0.

A weight/weight ratio of organic phosphorus stabilizers to hindered phenolic antioxidants may be from any one of about 9/1, about 8/1, about 7/1, about 6/1, about 5/1, about 4/1, about 3/1, about 2/1 or about 1/1 to any one of about 1/2, about 1/3, about 1/4, about 1/5, about 1/6, about 1/7, about 1/8 or about 1/9.

In certain embodiments, the compositions of the invention may be essentially free of organophosphorus stabilizers and/or hindered phenolic antioxidants. The term “essentially free” means “not intentionally added” and that only minor amounts may be present, for example levels of ≤100 ppm, ≤75 ppm, ≤50 ppm, s 25 ppm, ≤15 ppm, s 10 ppm, ≤5 ppm or S 2 ppm, by weight, based on the weight of the total composition.

The incorporation of the present benzofuranones and optional further additives into a polycarbonate is carried out by known methods, for example before or after molding or also by applying a dissolved or dispersed additive mixture to the polycarbonate, with or without subsequent evaporation of the solvent. Present benzofuranones and optional further additives can also be added to a polycarbonate in the form of a masterbatch which contains the additives in a concentration of, for example, about 2% to about 40% by weight, based on the total weight of the masterbatch. In the form of a masterbatch, the polymer of the masterbatch need not be the same as the polycarbonate. Molding may be carried out with known mixing machines, for instance mixers, kneaders or extruders.

Present benzofuranones and optional further additives can be premixed or added individually. Present benzofuranones and optional further additives can also be added before or during the polymerization or before crosslinking. Present benzofuranones and optional further additives can be incorporated into a polycarbonate in pure form or encapsulated in waxes, oils or polymers.

Present benzofuranones and optional further additives can also be sprayed onto a polycarbonate. They are able to dilute other additives or their melts so that they may be sprayed also together with these additives onto a polycarbonate.

In certain embodiments, the present benzofuranone additives are incorporated into a polycarbonate by melt blending in an extruder. The present benzofuranones and optional further additives may be added together or separately.

In some embodiments, additives may be incorporated into polycarbonate film or sheet (substrate) via coating processes on the film/sheet. “Dip coating” involves continuously drawing substrate through a liquid medium where additive(s) are adsorbed onto the surface or impregnated partially into the substrate. Such coating processes may allow higher concentrations of additives to be affixed to a substrate.

In some embodiments, a process of preparing a polycarbonate composition includes measurement of raw materials by weight followed by the dry blending of raw ingredients. The mixture may be fed to a screw extruder at a constant feed rate to extrude the material into a desired form, followed by water cooling and drying. Ingredients may be mixed and dosed via a single feeder at the throat or feeding zone of an extruder. Alternatively, some components may be dosed part way down an extruder barrel in a melting or mixing zone. For instance, liquid ingredients may be dosed in a later zone via liquid injection under pressure.

A typical flat-film or cast extrusion line for film or sheet generally comprises an extruder, a film or sheet die, a take-off and/or sheet cooling system and a wind-up or spooling system. For multi-layer sheet or film structures, either a single die with multiple exit ports or multiple extruders may feed molten polymers into suitably designed dies for multi-layer constructs. Optional processing of post-extruded film prior to wind up may include a stretch-draw apparatus to provide orientation of the film in machine, transverse or both directions in order to enhance certain mechanical film properties.

Following are some embodiments of the disclosure.

In a first embodiment, disclosed is a composition comprising a polycarbonate and one or more additives selected from the group consisting of benzofuranone stabilizers of formula I

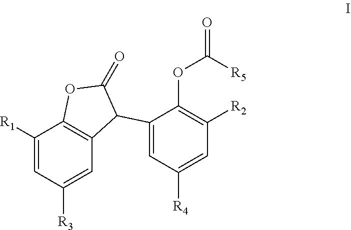

wherein R₁, R₂, R₃, R₄ and R₅ are each independently hydrogen, halogen, hydroxy, C₁-C₂₅alkyl, C₂-C₂₅alkenyl, C₂-C₂₅alkynyl, C₇-C₉phenylalkyl, phenyl, C₅-C₈cycloalkyl, C₁-C₁₈alkoxy, C₁-C₅alkylamine, di(C₁-C₅alkyl)amino, C₁-C₂₅alkanoyloxy, C₁-C₂₅alkanoylamino, C₃-C₂₅alkenoyloxy or C₃-C₂₅alkanoyloxy interrupted by one to three —O—, —S—, —NH— or —N(C-C₈alkyl)-, wherein Cr₁-C₂₅alkyl, C₂-C₂₅alkenyl, C₂-C₂₅alkynyl, C₇-C₉phenylalkyl, phenyl, C₅-C₈cycloalkyl, C₁-C₁₈alkoxy, C₁-C₈alkylamine, di(C₁-C₈alkyl)amino, Cr₁-C₂₅alkanoyloxy, C-C₂₅alkanoylamino, C₃-C₂₅alkenoyloxy and interrupted C₃-C₂₅alkenoyloxy are unsubstituted or substituted by one to three groups selected from halogen, hydroxy, thiol, amino, C-C₄alkylamino, di(C₁-C₄alkyl)amino and C₁-C₄alkyl.

In a second embodiment, in compounds of formula I, R₁ and R₂ are independently hydrogen or C₁-C₈alkyl, R₃ and R₄ are independently C₁-C₁₂alkyl and R₅ is C₁-C₇alkyl, phenyl, C₁-C₈alkylphenyl, 3,5-di(C₁-C₈alkyl)-4-hydroxyphenyl or C-C₈alkyl(3,5-di(C-C₈alkyl)-4-hydroxyphenyl); or wherein R₁ and R₂ are hydrogen and R₃ and R₄ are C-C₈alkyl. In a third embodiment, disclosed is a composition according to the first or second embodiments, wherein R₅ is C₁-C₄alkyl, phenyl, C₁-C₄alkylphenyl, 3,5-di(C₁-C₄alkyl)-4-hydroxyphenyl or C₁-C₄alkyl(3,5-di(C₁-C₄alkyl)-4-hydroxyphenyl. In a fourth embodiment, disclosed is a composition according to any of the preceding embodiments, wherein the benzofuranone stabilizers are selected from the group consisting of

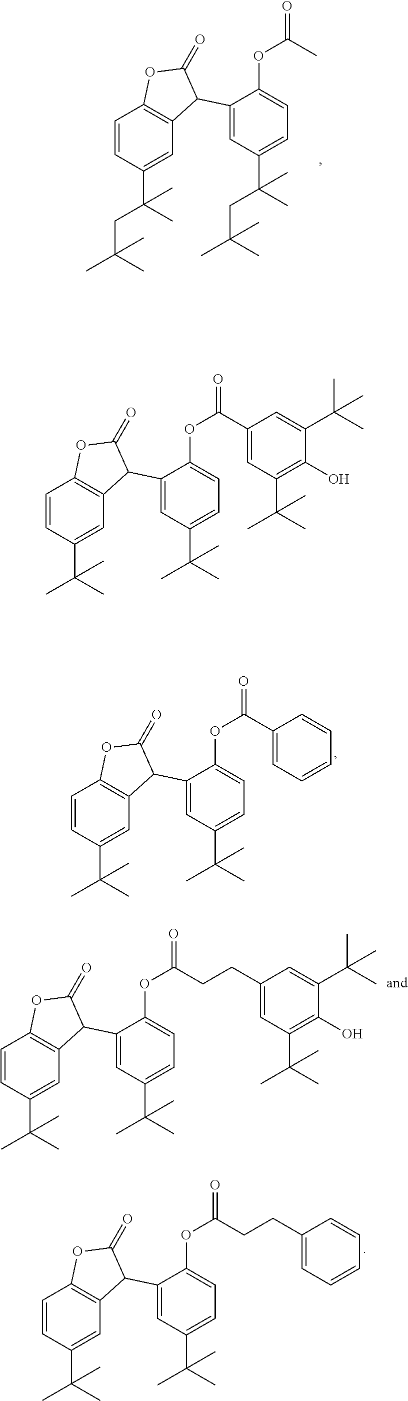 [SMILES: CC(=O)Oc1ccc(C(C)(C)CC(C)(C)C)cc1C1C(=O)Oc2ccc(C(C)(C)CC(C)(C)C)cc21 ; CC(C)(C)c1ccc(OC(=O)c2cc(C(C)(C)C)c(O)c(C(C)(C)C)c2)c(C2C(=O)Oc3ccc(C(C)(C)C)cc32)c1 ; CC(C)(C)c1ccc(OC(=O)c2ccccc2)c(C2C(=O)Oc3ccc(C(C)(C)C)cc32)c1 ; CC(C)(C)c1ccc(OC(=O)CCc2cc(C(C)(C)C)c(O)c(C(C)(C)C)c2)c(C2C(=O)Oc3ccc(C(C)(C)C)cc32)c1 ; CC(C)(C)c1ccc(OC(=O)CCc2ccccc2)c(C2C(=O)Oc3ccc(C(C)(C)C)cc32)c1]

In a fifth embodiment, disclosed is a composition according to any of the preceding embodiments, comprising from any one of about 10 ppm, about 25 ppm, about 50 ppm, about 100 ppm about 150 ppm, about 175 ppm about 200 ppm, about 225 ppm, about 250 ppm, about 275 ppm, about 300 ppm, about 325 ppm, about 350 ppm, about 400 ppm, about 450 ppm or about 500 ppm to any one of about 550 ppm, about 600 ppm, about 650 ppm, about 700 ppm, about 750 ppm, about 800 ppm, about 850 ppm, about 900 ppm, about 950 ppm or about 1000 ppm (0.1 weight percent (wt %)) by weight of the one or more benzofuranone stabilizers, based on the weight of the polycarbonate.

In a sixth embodiment, disclosed is a composition according to any of embodiments 1 to 4, comprising from any one of about 0.1 wt %, about 0.5 wt %, about 0.75 wt %, about 1.0 wt %, about 1.5 wt %, about 2.0 wt %, about 2.5 wt %, about 3.0 wt %, about 3.5 wt % or about 4.0 wt % to any one of about 4.5 wt %, about 5.0 wt %, about 5.5 wt %, about 6.0 wt %, about 6.5 wt % or about 7.0 wt % (weight percent) of the one or more benzofuranone stabilizers, based on the weight of the polycarbonate.

In a seventh embodiment, disclosed is a composition according to any of the preceding embodiments, comprising one or more organophosphorus stabilizers. In an eighth embodiment, disclosed is a composition according to any of the preceding embodiments, comprising one or more organophosphorus stabilizers selected from the group consisting of triphenyl phosphite, diphenyl alkyl phosphites, phenyl dialkyl phosphites, tris(nonylphenyl) phosphite, trilauryl phosphite, trioctadecyl phosphite, distearyl pentaerythritol diphosphite, tris(2,4-di-tert-butylphenyl) phosphite, bis(2,4-di-α-cumylphenyl) pentaerythrtitol diphosphite, diisodecyl pentaerythritol diphosphite, bis(2,4-di-tert-butylphenyl) pentaerythritol diphosphite, bis(2,6-di-tert-butyl-4-methylphenyl) pentaerythritol diphosphite, bisisodecyloxy-pentaerythritol diphosphite, bis(2,4-di-tert-butyl-6-methylphenyl) pentaerythritol diphosphite, bis(2,4,6-tri-tert-butylphenyl) pentaerythritol diphosphite, tristearyl sorbitol triphosphite, tetrakis (2,4-di-tert-butylphenyl) 4,4′-biphenylene-diphosphonite, 6-isooctyloxy-2,4,8,10-tetra-tert-butyl-dibenzo[d,f][1,3,2]dioxaphosphepin, 6-fluoro-2,4,8,10-tetra-tert-butyl-12-methyl-dibenzo[d,g][1,3,2]dioxaphosphocin, bis(2,4-di-tert-butyl-6-methylphenyl) methyl phosphite, bis(2,4-di-tert-butyl-6-methylphenyl) ethyl phosphite, 2,2′,2″-nitrilo[triethyltris(3,3′5,5′-tetra-tert-butyl-1,1′-biphenyl-2,2′-diyl) phosphite], bis(2,4-di-t-butylphenyl) octylphosphite, poly(4,4′-{2,2′-dimethyl-5,5′-di-t-butylphenylsulfide-}octylphosphite), poly(4,4′{-isopropylidenediphenol}-octylphosphite), poly(4,4′-{isopropylidenebis[2,6-dibromophenol]}-octylphosphite), poly(4,4′-{2,2′-dimethyl-5,5′-di-t-butylphenylsulfide}-pentaerythrityl diphosphite),

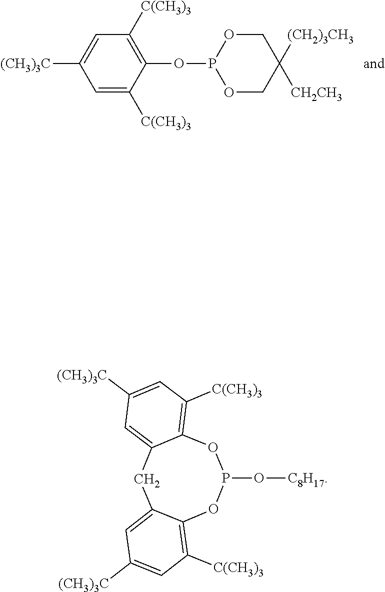

In a ninth embodiment, disclosed is a composition according to any of the preceding embodiments, comprising from any one of about 10 ppm, about 25 ppm, about 50 ppm, about 100 ppm about 150 ppm, about 175 ppm about 200 ppm, about 225 ppm, about 250 ppm, about 275 ppm, about 300 ppm, about 325 ppm, about 350 ppm, about 400 ppm, about 450 ppm or about 500 ppm to any one of about 550 ppm, about 600 ppm, about 650 ppm, about 700 ppm, about 750 ppm, about 800 ppm, about 850 ppm, about 900 ppm, about 950 ppm or about 1000 ppm (0.1 weight percent (wt %)) by weight of one or more organophosphorus stabilizers, based on the weight of the polycarbonate.

In a tenth embodiment, disclosed is a composition according to any of the preceding embodiments, wherein the composition comprises one or more benzofuranone stabilizers and one or more organophosphorus stabilizers, wherein a weight/weight ratio of the benzofuranone stabilizers to the organophosphorus stabilizers is from any one of about 1/99, about 1/90, about 1/80, about 1/70, about 1/60, about 1/50, about 1/40, about 1/30, about 1/20, about 1/10, about 1/9, about 1/8, about 1/7, about 1/6, about 1/5, about 1/4, about 1/3, about 1/2 or about 1/1 to any one of about 2/1, about 3/1, about 4/1, about 5/1, about 6/1, about 7/1, about 8/1, about 9/1, about 10/1, about 20/1, about 30/1, about 40/1, about 50/1, about 60/1, about 70/1, about 80/1, about 90/1 or about 99/1.

In an eleventh embodiment, disclosed is a composition according to any of the preceding embodiments, wherein the composition comprises one or more benzofuranone stabilizers and one or more organophosphorous stabilizers, wherein a weight/weight ratio of the one or more benzofuranone stabilizers to the one or more organophosphorus stabilizers may be from any one of about 0.05, about 0.10, about 0.15, about 0.20, about 0.25, about 0.30, about 0.35, about 0.40, about 0.45 or about 0.50 to any one of about 0.55, about 0.60, about 0.65, about 0.70, about 0.75, about 0.80, about 0.85, about 0.90, about 0.95 or about 1.0.

In a twelfth embodiment, disclosed is a composition according to any of embodiments 1 to 6, wherein the composition is essentially free of organophosphorus stabilizers. In a thirteenth embodiment, disclosed is a composition according to any of the preceding embodiments, wherein the composition is essentially free of hindered phenolic antioxidants.

In a fourteenth embodiment, disclosed is a composition according to any of the preceding embodiments, wherein the composition is transparent to light having a wavelength of from any of about 350 nm, about 360 nm, about 370 nm, about 380 nm, about 390 nm, about 400 nm, about 410 nm, about 420 nm, about 430 nm, about 440 nm or about 450 nm to about 700 nm.

In a fifteenth embodiment, disclosed is a film, sheet or molded article comprising the composition according to any of the preceding embodiments. In a sixteenth embodiment, disclosed is a film, sheet or molded article according to the fifteenth embodiment, which is transparent to light having a wavelength of from about 350 nm to about 700 nm.

In an seventeenth embodiment, disclosed is a film, sheet or molded article according to embodiments 15 or 16, which is selected from the group consisting of automotive headlamp covers, street lamp covers, solar energy control films, reflective housings for lighting fixtures and light diffuser films.

In an eighteenth embodiment, disclosed is a method of preparing the film, sheet or molded article according to any of embodiments 15 to 17, the method comprising melt blending the composition.

In a nineteenth embodiment, disclosed is a method of stabilizing polycarbonate against the deleterious effects of heat, light and oxygen, the method comprising incorporating into a polycarbonate one or more additives selected from the group consisting of benzofuranone stabilizers of formula I

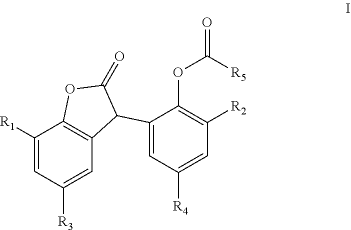

wherein R₁ and R₂ are independently hydrogen or C₁-C₈alkyl, R₃ and R₄ are independently C₁-C₁₂alkyl and R₅ is C₁-C₇alkyl, phenyl, C₁-C₈alkylphenyl, 3,5-di(C₁-C₈alkyl)-4-hydroxyphenyl or C₁-C₈alkyl(3,5-di(C₁-C₈alkyl)-4-hydroxyphenyl). In a twentieth embodiment, disclosed is a method according to embodiment 19, wherein R₁ and R₂ are hydrogen and R₃ and R₄ are C₁-C₈alkyl. In a twenty first embodiment, disclosed is a method according to embodiments 19 or 20, wherein R₅ is C₁-C₄alkyl, phenyl, C₁-C₄alkylphenyl, 3,5-di(C₁-C₄alkyl)-4-hydroxyphenyl or C₁-C₄alkyl(3,5-di(C₁-C₄alkyl)-4-hydroxyphenyl.

In a twenty second embodiment, disclosed is a method according to any of embodiments 19 to 21, wherein the benzofuranone stabilizers are selected from the group consisting of

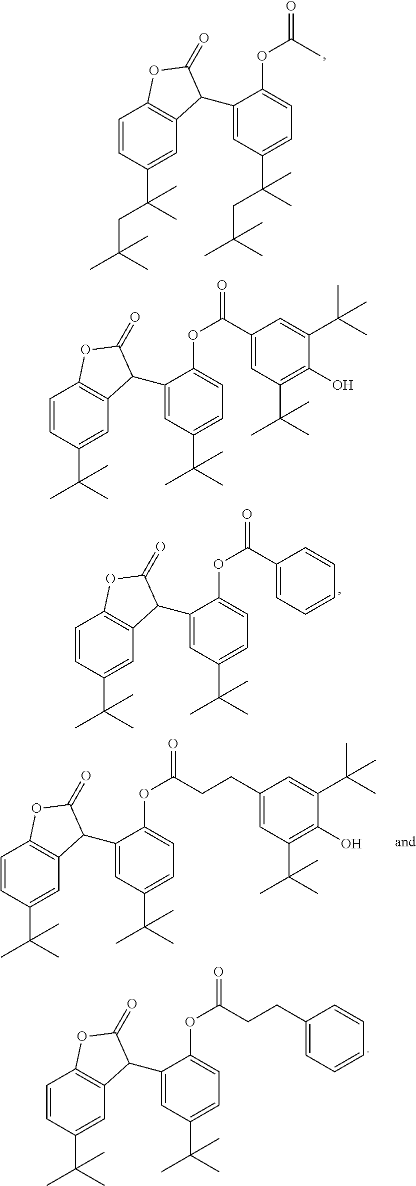

In a twenty third embodiment, disclosed is a method according to any of embodiments 19 to 22, comprising incorporating from any one of about 10 ppm, about 25 ppm, about 50 ppm, about 100 ppm about 150 ppm, about 175 ppm about 200 ppm, about 225 ppm, about 250 ppm, about 275 ppm, about 300 ppm, about 325 ppm, about 350 ppm, about 400 ppm, about 450 ppm or about 500 ppm to any one of about 550 ppm, about 600 ppm, about 650 ppm, about 700 ppm, about 750 ppm, about 800 ppm, about 850 ppm, about 900 ppm, about 950 ppm or about 1000 ppm (0.1 weight percent (wt %)) by weight of the one or more benzofuranone stabilizers, based on the weight of the polycarbonate.

In a twenty fourth embodiment, disclosed is a method according to any of embodiments 19 to 23, comprising incorporating from any one of about 0.1 wt %, about 0.5 wt %, about 0.75 wt %, about 1.0 wt %, about 1.5 wt %, about 2.0 wt %, about 2.5 wt %, about 3.0 wt %, about 3.5 wt % or about 4.0 wt % to any one of about 4.5 wt %, about 5.0 wt %, about 5.5 wt %, about 6.0 wt %, about 6.5 wt % or about 7.0 wt % (weight percent) of the one or more benzofuranone stabilizers, based on the weight of the polycarbonate.

In a twenty fifth embodiment, disclosed is a method according to any of embodiments 19 to 24, comprising incorporating one or more organophosphorus stabilizers. In a twenty sixth embodiment, disclosed is a method according to any of embodiments 19 to 25, comprising incorporating one or more organophosphorus stabilizers selected from the group consisting of triphenyl phosphite, diphenyl alkyl phosphites, phenyl dialkyl phosphites, tris(nonylphenyl) phosphite, trilauryl phosphite, trioctadecyl phosphite, distearyl pentaerythritol diphosphite, tris(2,4-di-tert-butylphenyl) phosphite, bis(2,4-di-α-cumylphenyl) pentaerythrtitol diphosphite, diisodecyl pentaerythritol diphosphite, bis(2,4-di-tert-butylphenyl) pentaerythritol diphosphite, bis(2,6-di-tert-butyl-4-methylphenyl) pentaerythritol diphosphite, bisisodecyloxy-pentaerythritol diphosphite, bis(2,4-di-tert-butyl-6-methylphenyl) pentaerythritol diphosphite, bis(2,4,6-tri-tert-butylphenyl) pentaerythritol diphosphite, tristearyl sorbitol triphosphite, tetrakis (2,4-di-tert-butylphenyl) 4,4′-biphenylene-diphosphonite, 6-isooctyloxy-2,4,8,10-tetra-tert-butyl-dibenzo[d,f][1,3,2]dioxaphosphepin, 6-fluoro-2,4,8,10-tetra-tert-butyl-12-methyl-dibenzo[d,g][1,3,2]dioxaphosphocin, bis(2,4-di-tert-butyl-6-methylphenyl) methyl phosphite, bis(2,4-di-tert-butyl-6-methylphenyl) ethyl phosphite, 2,2′,2″-nitrilo[triethyltris(3,3′5,5′-tetra-tert-butyl-1,1′-biphenyl-2,2′-diyl) phosphite], bis(2,4-di-t-butylphenyl) octylphosphite, poly(4,4′-{2,2′-dimethyl-5,5′-di-t-butylphenylsulfide-}octylphosphite), poly(4,4′{-isopropylidenediphenol}-octylphosphite), poly(4,4′-{isopropylidenebis[2,6-dibromophenol]}-octylphosphite), poly(4,4′-{2,2′-dimethyl-5,5′-di-t-butylphenylsulfide}-pentaerythrityl diphosphite),

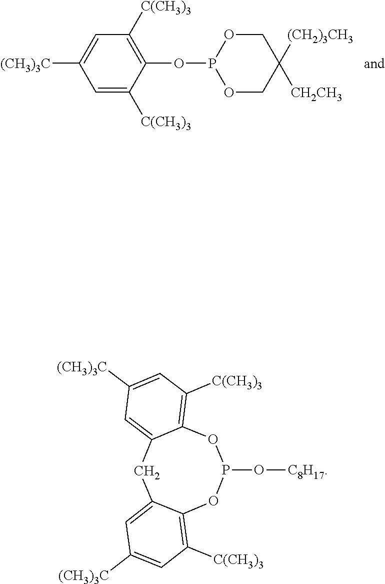

In a twenty seventh embodiment, disclosed is a method according to any of embodiments 19 to 26, comprising incorporating from any one of about 10 ppm, about 25 ppm, about 50 ppm, about 100 ppm about 150 ppm, about 175 ppm about 200 ppm, about 225 ppm, about 250 ppm, about 275 ppm, about 300 ppm, about 325 ppm, about 350 ppm, about 400 ppm, about 450 ppm or about 500 ppm to any one of about 550 ppm, about 600 ppm, about 650 ppm, about 700 ppm, about 750 ppm, about 800 ppm, about 850 ppm, about 900 ppm, about 950 ppm or about 1000 ppm (0.1 weight percent (wt %)) by weight of one or more organophosphorus stabilizers, based on the weight of the polycarbonate.

In a twenty eighth embodiment, disclosed is a method according to any of embodiments 19 to 27, comprising incorporating one or more benzofuranone stabilizers and one or more organophosphorus stabilizers, wherein a weight/weight ratio of the benzofuranone stabilizers to the organophosphorus stabilizers is from any one of about 1/99, about 1/90, about 1/80, about 1/70, about 1/60, about 1/50, about 1/40, about 1/30, about 1/20, about 1/10, about 1/9, about 1/8, about 1/7, about 1/6, about 1/5, about 1/4, about 1/3, about 1/2 or about 1/1 to any one of about 2/1, about 3/1, about 4/1, about 5/1, about 6/1, about 7/1, about 8/1, about 9/1, about 10/1, about 20/1, about 30/1, about 40/1, about 50/1, about 60/1, about 70/1, about 80/1, about 90/1 or about 99/1.

In a twenty ninth embodiment, disclosed is a method according to any of embodiments 19 to 28, comprising incorporating one or more benzofuranone stabilizers and one or more organophosphorous stabilizers, wherein a weight/weight ratio of the one or more benzofuranone stabilizers to the one or more organophosphorus stabilizers may be from any one of about 0.05, about 0.10, about 0.15, about 0.20, about 0.25, about 0.30, about 0.35, about 0.40, about 0.45 or about 0.50 to any one of about 0.55, about 0.60, about 0.65, about 0.70, about 0.75, about 0.80, about 0.85, about 0.90, about 0.95 or about 1.0.

In a thirtieth embodiment, disclosed is a method according to any of embodiments 19 to 24, wherein the method does not comprise incorporating organophosphorus stabilizers. In a thirty first embodiment, disclosed is a method according to any of embodiments 19 to 30, wherein the method does not comprise incorporating hindered phenolic antioxidants.

In a thirty second embodiment, disclosed is an additive composition comprising one or more benzofuranone stabilizers of formula I

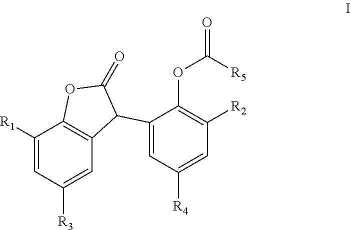

wherein R₁ and R₂ are independently hydrogen or C₁-C₈alkyl, R₃ and R₄ are independently C₁-C₁₂alkyl and R₅ is C₁-C₇alkyl, phenyl, C₁-C₈alkylphenyl, 3,5-di(C₁-C₈alkyl)-4-hydroxyphenyl or C₁-C₈alkyl(3,5-di(C₁-C₈alkyl)-4-hydroxyphenyl) and one or more organophosphorus stabilizers.

In a thirty third embodiment, disclosed is a composition according to the thirty second embodiment, wherein R₁ and R₂ are hydrogen and R₃ and R₄ are C₁-C₈alkyl. In a thirty fourth embodiment, disclosed is a composition according to embodiments 32 or 33, wherein R₅ is C₁-C₄alkyl, phenyl, C₁-C₄alkylphenyl, 3,5-di(C₁-C₄alkyl)-4-hydroxyphenyl or C₁-C₄alkyl(3,5-di(C₁-C₄alkyl)-4-hydroxyphenyl.

In a thirty fifth embodiment, disclosed is a composition according to any of embodiments 33 to 34, wherein the benzofuranone stabilizers are selected from the group consisting of

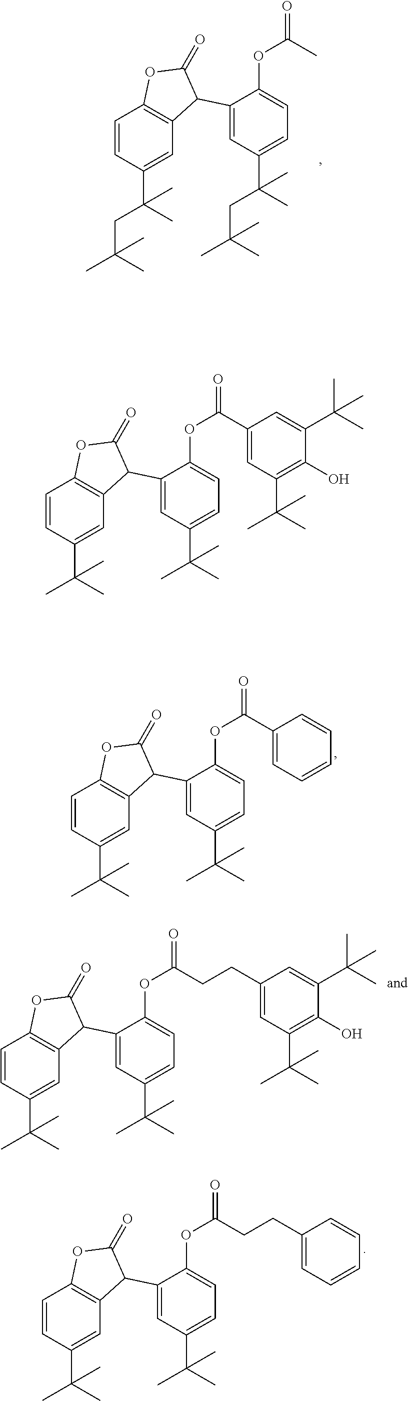 [SMILES: CC(=O)Oc1ccc(C(C)(C)CC(C)(C)C)cc1C1C(=O)Oc2ccc(C(C)(C)CC(C)(C)C)cc21 ; CC(C)(C)c1ccc(OC(=O)c2cc(C(C)(C)C)c(O)c(C(C)(C)C)c2)c(C2C(=O)Oc3ccc(C(C)(C)C)cc32)c1 ; CC(C)(C)c1ccc(OC(=O)c2ccccc2)c(C2C(=O)Oc3ccc(C(C)(C)C)cc32)c1 ; CC(C)(C)c1ccc(OC(=O)CCc2cc(C(C)(C)C)c(O)c(C(C)(C)C)c2)c(C2C(=O)Oc3ccc(C(C)(C)C)cc32)c1 ; CC(C)(C)c1ccc(OC(=O)CCc2ccccc2)c(C2C(=O)Oc3ccc(C(C)(C)C)cc32)c1]

In a thirty sixth embodiment, disclosed is a composition according to any of embodiments 32 to 35, comprising one or more organophosphorus stabilizers selected from the group consisting of triphenyl phosphite, diphenyl alkyl phosphites, phenyl dialkyl phosphites, tris(nonylphenyl) phosphite, trilauryl phosphite, trioctadecyl phosphite, distearyl pentaerythritol diphosphite, tris(2,4-di-tert-butylphenyl) phosphite, bis(2,4-di-α-cumylphenyl) pentaerythrtitol diphosphite, diisodecyl pentaerythritol diphosphite, bis(2,4-di-tert-butylphenyl) pentaerythritol diphosphite, bis(2,6-di-tert-butyl-4-methylphenyl) pentaerythritol diphosphite, bisisodecyloxy-pentaerythritol diphosphite, bis(2,4-di-tert-butyl-6-methylphenyl) pentaerythritol diphosphite, bis(2,4,6-tri-tert-butylphenyl) pentaerythritol diphosphite, tristearyl sorbitol triphosphite, tetrakis (2,4-di-tert-butylphenyl) 4,4′-biphenylene-diphosphonite, 6-isooctyloxy-2,4,8,10-tetra-tert-butyl-dibenzo[d,f][1,3,2]dioxaphosphepin, 6-fluoro-2,4,8,10-tetra-tert-butyl-12-methyl-dibenzo[d,g][1,3,2]dioxaphosphocin, bis(2,4-di-tert-butyl-6-methylphenyl) methyl phosphite, bis(2,4-di-tert-butyl-6-methylphenyl) ethyl phosphite, 2,2′,2″-nitrilo[triethyltris(3,3′5,5′-tetra-tert-butyl-1,1′-biphenyl-2,2′-diyl) phosphite], bis(2,4-di-t-butylphenyl) octylphosphite, poly(4,4′-{2,2′-dimethyl-5,5′-di-t-butylphenylsulfide-}octylphosphite), poly(4,4′{-isopropylidenediphenol}-octylphosphite), poly(4,4′-{isopropylidenebis[2,6-dibromophenol]}-octylphosphite), poly(4,4′-{2,2′-dimethyl-5,5′-di-t-butylphenylsulfide}-pentaerythrityl diphosphite),

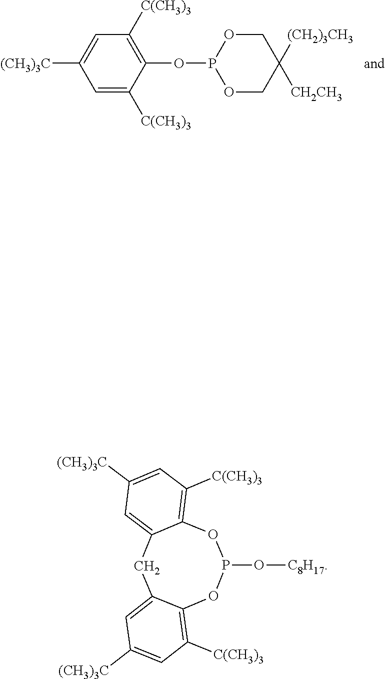

In a thirty seventh embodiment, disclosed is a composition according to any of embodiments 32 to 36, wherein a weight/weight ratio of the benzofuranone stabilizers to the organophosphorus stabilizers is from any one of about 1/99, about 1/90, about 1/80, about 1/70, about 1/60, about 1/50, about 1/40, about 1/30, about 1/20, about 1/10, about 1/9, about 1/8, about 1/7, about 1/6, about 1/5, about 1/4, about 1/3, about 1/2 or about 1/1 to any one of about 2/1, about 3/1, about 4/1, about 5/1, about 6/1, about 7/1, about 8/1, about 9/1, about 10/1, about 20/1, about 30/1, about 40/1, about 50/1, about 60/1, about 70/1, about 80/1, about 90/1 or about 99/1.

In a thirty eighth embodiment, disclosed is a composition according to any of embodiments 32 to 36, wherein a weight/weight ratio of the one or more benzofuranone stabilizers to the one or more organophosphorus stabilizers may be from any one of about 0.05, about 0.10, about 0.15, about 0.20, about 0.25, about 0.30, about 0.35, about 0.40, about 0.45 or about 0.50 to any one of about 0.55, about 0.60, about 0.65, about 0.70, about 0.75, about 0.80, about 0.85, about 0.90, about 0.95 or about 1.0.

In a thirty ninth embodiment, disclosed is a composition according to any of embodiments 32 to 38, wherein the composition is essentially free of hindered phenolic antioxidants.

The articles “a” and “an” herein refer to one or to more than one (e.g. at least one) of the grammatical object. Any ranges cited herein are inclusive. The term “about” used throughout is used to describe and account for small fluctuations. For instance, “about” may mean the numeric value may be modified by ±5%, ±4%, ±3%, ±2%, ±1%, ±0.5%, ±0.4%, ±0.3%, ±0.2%, ±0.1% or ±0.05%. All numeric values are modified by the term “about” whether or not explicitly indicated. Numeric values modified by the term “about” include the specific identified value. For example “about 5.0” includes 5.0.

U.S. patents, U.S. patent applications and published U.S. patent applications discussed herein are hereby incorporated by reference.

Unless otherwise indicated, all parts and percentages are by weight. Weight percent (wt %), if not otherwise indicated, is based on an entire composition free of any volatiles.

### EXAMPLES

The polycarbonate employed in the Examples is LEXAN 141; which is determined to contain from about 600 ppm to about 650 ppm tris(2,4-di-t-butylphenyl)phosphite and from about 250 ppm to about 300 ppm of tris(2,4-di-t-butylphenyl)phosphate as commercially obtained. The additives employed in the Examples below are: phosphite: tris(2,4-di-t-butylphenyl)phosphite; hindered phenol: octadecyl 3-(3,5-di-tert-butyl-4-hydroxyphenyl)propionate; triazine A: 2,4-bis(4-biphenylyl)-6-(2-hydroxy-4-(2-ethylhexyl)oxy-phenyl)-s-triazine; triazine B: 4,6-diphenyl-2-(4-hexyloxy-2-hydroxyphenyl)-s-triazine; benzotriazole A: 2-(2-hydroxy-5-t-octylphenyl)-2H-benzotriazole; benzotriazole B: 2,2′-methylene-bis(4-t-octyl-(6-2H-benzotriazol-2-yl)phenol); TPP: triphenylphosphine;

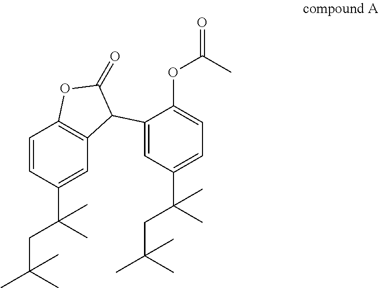 [SMILES: CC(=O)Oc1ccc(C(C)(C)CC(C)(C)C)cc1C1C(=O)Oc2ccc(C(C)(C)CC(C)(C)C)cc21]

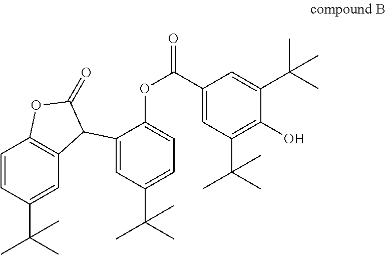 [SMILES: CC(C)(C)c1ccc(OC(=O)c2cc(C(C)(C)C)c(O)c(C(C)(C)C)c2)c(C2C(=O)Oc3ccc(C(C)(C)C)cc32)c1]

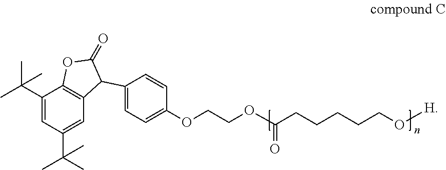

Compound C is disclosed for example in U.S. Pat. No. 7,390,912, wherein n=4.

### Example 1 Oven Aging

Additives are dry blended with polycarbonate to provide the following formulations. Additive levels are reported in ppm (parts-per-million) by weight, based on the weight of polycarbonate.

| sample | additive(s) |
|---|---|
| 1 | — |
| 2 | 800 ppm phosphite/200 ppm hindered phenol |
| 3 | 200 ppm compound A |
| 4 | 400 ppm compound A |
| 5 | 200 ppm compound B |
| 6 | 400 ppm compound A/600 ppm phosphite |
| 7 | 600 ppm compound A/400 ppm phosphite |
| 8 | 200 ppm compound B/600 ppm phosphite |
| 9 | 600 ppm compound B/400 ppm phosphite |

The admixed polycarbonate formulations are injection molded into 2×2×0.125 inch plaques. The plaques are subjected to oven aging at 125° C. and are monitored up to 12,000 hours. Haze is determined on an average of 3 plaques. Yellowness index (YI), delta E (AE), transmission (T) and haze determined after 12,000 hours oven ageing at 125° C. for plaques containing samples 1-9 are reported below.

| sample | YI | ΔE | T | haze |
|---|---|---|---|---|
| 1 | 25.1 | 14.8 | 80.6 | 17.6 |
| 2 | 27.0 | 13.4 | 79.4 | 10.8 |
| 3 | 15.9 | 6.3 | 83.0 | 8.7 |
| 4 | 17.5 | 6.9 | 83.4 | 9.9 |
| 5 | 17.7 | 7.8 | 79.0 | 8.6 |
| 6 | 18.0 | 7.9 | 81.9 | 10.9 |
| 7 | 21.2 | 9.8 | 82.0 | 11.3 |
| 8 | 15.6 | 5.9 | 84.0 | 11.8 |
| 9 | 21.6 | 10.4 | 81.8 | 10.9 |

### Example 2 Oven Aging

Additives are dry blended with polycarbonate to provide the following formulations. Additive levels are reported in ppm by weight, based on the weight of polycarbonate.

| sample | additive(s) |
|---|---|
| 1 | — |
| 2 | 320 ppm phosphite/80 ppm hindered phenol |
| 3 | 200 ppm compound A |
| 4 | 400 ppm compound A |
| 5 | 600 ppm compound A |
| 6 | 400 ppm compound B |
| 7 | 400 ppm compound C |
| 8 | 600 ppm compound C |
| 9 | 200 ppm compound A/400 ppm phosphite |
| 10 | 400 ppm compound A/400 ppm phosphite |
| 11 | 600 ppm compound A/400 ppm phosphite |
| 12 | 400 ppm compound B/400 ppm phosphite |
| 13 | 400 ppm compound C/400 ppm phosphite |
| 14 | 600 ppm compound C/400 ppm phosphite |
| 15 | 5000 ppm triazine A |
| 16 | 400 ppm compound A/5000 ppm triazine A |
| 17 | 600 ppm compound A/5000 ppm triazine A |
| 18 | 400 ppm compound B/5000 ppm triazine A |
| 19 | 400 ppm compound C/5000 ppm triazine A |
| 20 | 600 ppm compound C/5000 ppm triazine A |

The admixed polycarbonate formulations are injection molded into 2×2×0.125 inch plaques. The plaques are subjected to oven aging at 125° C. Yellowness Index (YI) is measured at zero hours and at 5055 hours. Delta YI (ΔYI) is YI at 5055 hours minus YI at zero hours. Results are below.

| sample | 0 hours | 5055 hours | ΔYI |
|---|---|---|---|
| 1 | 6.7 | 36.3 | 29.6 |
| 2 | 6.9 | 29.2 | 22.3 |
| 3 | 7.3 | 17.5 | 10.2 |
| 4 | 7.3 | 15.5 | 8.2 |
| 5 | 6.0 | 14.4 | 8.4 |
| 6 | 6.2 | 15.0 | 8.8 |
| 7 | 6.6 | 17.1 | 10.4 |
| 8 | 6.0 | 16.3 | 10.3 |
| 9 | 5.1 | 15.2 | 10.1 |
| 10 | 5.0 | 13.9 | 8.9 |
| 11 | 4.8 | 13.2 | 8.4 |
| 12 | 5.1 | 14.7 | 9.6 |
| 13 | 5.0 | 14.4 | 9.4 |
| 14 | 4.9 | 15.4 | 10.5 |
| 15 | 14.3 | 35.3 | 21.0 |
| 16 | 14.9 | 22.3 | 7.4 |
| 17 | 14.6 | 21.6 | 6.9 |
| 18 | 14.4 | 22.1 | 7.7 |
| 19 | 16.5 | 32.9 | 16.4 |
| 20 | 14.6 | 24.0 | 9.4 |

### Example 3 Accelerated Light Aging

Additives are dry blended with polycarbonate to provide the following formulations. Additive levels are in ppm by weight, based on the weight of polycarbonate.

| sample | additive(s) |
|---|---|
| 1 | — |
| 2 | 600 ppm compound A/3000 ppm triazine A/400 ppm phosphite |
| 3 | 600 ppm compound A/3000 ppm triazine B/400 ppm phosphite |
| 4 | 600 ppm compound A/3000 ppm benzotriazole A/400 ppm |
| | phosphite |
| 5 | 600 ppm compound A/3000 ppm benzotriazole B/400 ppm |
| | phosphite |
| 6 | 600 ppm compound B/3000 ppm triazine B/400 ppm phosphite |
| 7 | 600 ppm compound B/3000 ppm benzotriazole A/400 ppm |
| | phosphite |
| 8 | 600 ppm compound A/3000 ppm triazine B/400 ppm TPP |
| 9 | 600 ppm compound A/3000 ppm benzotriazole A/400 ppm TPP |

The admixed polycarbonate formulations are injection molded into 2×2×0.125 inch plaques. The plaques are subjected to accelerated weathering in a xenon-arc Weather-O-Meter. Yellowness Index (YI) is measured at zero hours and at 3022 hours. Delta YI (ΔYI) is YI at 3022 hours minus YI at zero hours. Results are below.

| sample | 0 hours | 3022 hours | ΔYI |
|---|---|---|---|
| 1 | 5.1 | 35.8 | 30.6 |
| 2 | 7.4 | 15.9 | 8.5 |
| 3 | 6.3 | 15.5 | 9.2 |
| 4 | 2.9 | 15.1 | 12.2 |
| 5 | 3.4 | 16.9 | 13.5 |
| 6 | 5.2 | 15.3 | 10.1 |
| 7 | 2.8 | 14.8 | 12.0 |
| 8 | 4.7 | 14.8 | 10.1 |
| 9 | 2.4 | 14.6 | 12.2 |

1-14. (canceled)

15. A film, sheet or molded article comprising a composition comprising a polycarbonate and one or more additives selected from the group consisting of benzofuranone stabilizers of formula I: 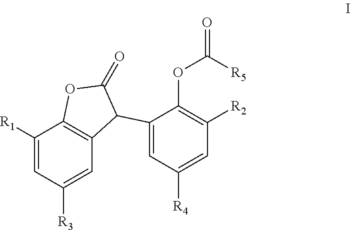
wherein:
R₁, R₂, R₃, R₄ and R₅ are each independently hydrogen, halogen, hydroxy, C₁-C₂₅alkyl, C₂-C₂₅alkenyl, C₂-C₂₅alkynyl, C₇-C₉phenylalkyl, phenyl, C₅-C₂₅cycloalkyl, C₁-C₁₈alkoxy, C₁-C₈alkylamine, di(C₁-C₈alkyl)amino, C₁-C₂5alkanoyloxy, C₁-C₂₅alkanoylamino, C₃-C₂₅alkenoyloxy or C₃-C₂₅alkanoyloxy interrupted by one to three —O—, —S—, —NH— or —N(C₁-C₈alkyl)-; and
C₁-C₂₅alkyl, C₂-C₂₅alkenyl, C₂-C₂₅alkynyl, C₇-C₉phenylalkyl, phenyl, C₅-C₈cycloalkyl, C₁-C₁₅alkoxy, C₁-C₈alkylamine, di(C₁-C₈alkyl)amino, C₁-C₂₅alkanoyloxy, C₁-C₂₅alkanoylamino, C₃-C₂₅alkenoyloxy, and interrupted C₃-C₂₅alkenoyloxy are unsubstituted or substituted by one to three groups selected from the group consisting of halogen, hydroxy, thiol, amino, C₁-C₄alkylamino di(C₁-C₄alkyl)amino, and C₁-C₄alkyl.

16. The film, sheet or molded article according to claim 15, which is transparent to light having a wavelength of from about 400 nm to about 700 nm.

17. The film, sheet or molded article according to claim 15, which is selected from the group consisting of automotive headlamp covers, street lamp covers, solar energy control films, reflective housings for lighting fixtures and light diffuser films.

18. A method of preparing the film, sheet or molded article according to claim 15, the method comprising melt blending the composition.

19. A method of stabilizing polycarbonate against the deleterious effects of heat, light and oxygen, the method comprising incorporating into a polycarbonate one or more additives selected from the group consisting of benzofuranone stabilizers of formula I: 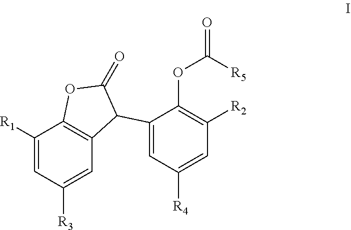
wherein:
R₁, R₂, R₃, R₄ and R₅ are each independently hydrogen, halogen, hydroxy, C₁-C₂₅alkyl, C₂-C₂₅alkenyl, C₂-C₂₅alkynyl, C₇-C₉phenylalkyl, phenyl, C₅-C₈cycloalkyl, C₁-C₁₈alkoxy, C₁-C₅alkylamine, di(C₁-C₈alkyl)amino, C₁-C₂₅alkanoyloxy, C₁-C₂₅alkanoylamino, C₃-C₂₅alkenoyloxy or C₃-C₂₅alkanoyloxy interrupted by one to three —O—, —S—, —NH— or —N(C₁-C₈alkyl)-; and
C₁-C₂₅alkyl, C₂-C₂₅alkenyl, C₂-C₂₅alkynyl, C₇-C₉phenylalkyl, phenyl, C₅-C₈cycloalkyl, C₁-C₁₈alkoxy, C₁-C₈alkylamine, di(C₁-C₈alkyl)amino, C₁-C₂₅alkanoyloxy, C₁-C₂₅alkanoylamino, C₃-C₂₅alkenoyloxy, and interrupted C₃-C₂₅alkenoyloxy are unsubstituted or substituted by one to three groups selected from the group consisting of halogen, hydroxy, thiol, amino, C₁-C₄alkylamino di(C₁-C₄alkyl)amino, and C₁-C₄alkyl.

20. An additive composition comprising one or more organophosphorus stabilizers and one or more benzofuranone stabilizers of formula I: 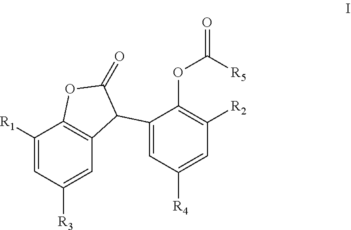
wherein:
R₁, R₂, R₃, R₄ and R₅ are each independently hydrogen, halogen, hydroxy, C₁-C₂₅alkyl, C₂-C₂₅alkenyl, C₂-C₂₅alkynyl, C₇-C₉phenylalkyl, phenyl, C₅-C₈cycloalkyl, C₁-C₁₈alkoxy, C₁-C₈alkylamine, di(C₁-C₈alkyl)amino, C₁-C₂₅alkanoyloxy, C₁-C₂₅alkanoylamino, C₃-C₂₅alkenoyloxy or C₃-C₂₅alkanoyloxy interrupted by one to three —O—, —S—, —NH— or —N(C₁-C₈alkyl)-; and
C₁-C₂₅alkyl, C₂-C₂₅alkenyl, C₂-C₂₅alkynyl, C₇-C₉phenylalkyl, phenyl, C₅-C₈cycloalkyl, C₁-C₁₅alkoxy, C₁-C₈alkylamine, di(C₁-C₈alkyl)amino, C₁-C₂₅alkanoyloxy, C₁-C₂₅alkanoylamino, C₃-C₂₅alkenoyloxy, and interrupted C₃-C₂₅alkenoyloxy are unsubstituted or substituted by one to three groups selected from the group consisting of halogen, hydroxy, thiol, amino, C₁-C₄alkylamino di(C₁-C₄alkyl)amino, and C₁-C₄alkyl.

21. The composition according to claim 20, wherein a weight/weight ratio of the one or more benzofuranone stabilizers to the one or more organophosphorus stabilizers is from about 0.05 to about 1.0.

22. The composition according to claim 20, wherein the composition is essentially free of hindered phenolic antioxidants.

23. The composition according to claim 20, wherein the composition comprises one or more ultraviolet light absorbers.
